(19) United States
(12) Patent Application Publication
(10) Pub. No.: US 2016/0138098 A1
(43) Pub. Date: May 19, 2016

(54) CONTAINER ACCOMMODATION BODY

(71) Applicant: Seiko Epson Corporation, Tokyo (JP)

(72) Inventors: Kotaro IDEGAMI, Chino (JP); Masato HANAMURA, Shiojiri (JP)

(21) Appl. No.: 14/939,230

(22) Filed: Nov. 12, 2015

(30) Foreign Application Priority Data
Nov. 17, 2014 (JP) 2014-232504

(51) Int. Cl. C12Q 1/68; B01L 3/00

(52) U.S. Cl. CPC C12Q 1/6848; B01L 3/52; C12Q 1/686; B01L 2200/16; B01L 2300/0832

(57) ABSTRACT

A container accommodation body includes: a package; a container that is accommodated and sealed in the package while accommodating and sealing a reagent for performing a nucleic acid amplification reaction; and a drying agent that is accommodated and sealed in the package.

### BACKGROUND

1. Technical Field

The present invention relates to a container accommodation body.

2. Related Art

Polymerase chain reaction (PCR) technology is established in the field of biochemistry. In recent years, the amplification accuracy or the detection sensitivity of a PCR method has improved, and the PCR method has progressed such that a very small amount of a specimen (DNA or the like) is capable of being amplified, detected, and analyzed. PCR is a technique of amplifying target nucleic acid by causing a solution (reaction solution) containing nucleic acid (target nucleic acid) (which is a target for amplification) and a reagent to undergo thermal cycling. Typically, the thermal cycling of the PCR is performed while temperature is adjusted in two stages or three stages.

In contrast, actually, a simplified test kit such as immunochromatography is mainly used to diagnose an infectious disease such as influenza in the medical field. However, the accuracy of such a simplified test may not be satisfactory, and the PCR is desirably adopted to diagnose an infectious disease such that higher test accuracy is capable of being anticipated.

In recent years, a device used in the PCR method or the like, which purifies nucleic acid by alternatingly stacking aqueous liquid layers and water-insoluble gel layers on top of each other in a capillary (cartridge), and causing a magnetic particle to pass through the layers, with nucleic acid adhering thereto has been proposed (refer to International Publication No. 2012-086243). In the method disclosed in International Publication No. 2012-086243, alcohol is used as a cleaning solution for cleaning the magnetic particle to which the nucleic acid adheres, and water is used as an elution solution for eluting the nucleic acid from the magnetic particle.

However, for example, when the device is stored for a long time, water contained in the cleaning solution, the elution solution, or the like, or water content or the like in the atmosphere may diffuse, and come into contact with a reagent (for performing a nucleic acid amplification reaction) that is accommodated in a reaction container. As a result, the PCR may be restricted.

### SUMMARY

An advantage of some aspects of the invention is to provide a container accommodation body capable of restricting water from coming into contact with a reagent for performing a nucleic acid amplification reaction even if the container accommodation body is stored for a long time.

### Application Example 1

A container accommodation body according to this application example includes: a package; a container that is accommodated and sealed in the package while accommodating and sealing a reagent for performing a nucleic acid amplification reaction; and a drying agent that is accommodated and sealed in the package.

According to the container accommodation body of this application example, even if water enters the package, since the drying agent absorbs the water, the water is capable of being restricted from coming into contact with the reagent. Accordingly, even if the container accommodation body according to the application example is stored for a long time, water is capable of being restricted from coming into contact with the reagent for performing the nucleic acid amplification reaction.

### Application Example 2

In the container accommodation body according to the application example, the package may have water permeability that is lower than that of the container.

According to the container accommodation body of this application example, water contained in the cleaning solution or the elution solution, or water content or the like in the atmosphere is capable of being restricted from entering the reaction container and coming into contact with the reagent compared to when the container is not accommodated and sealed in a package.

### Application Example 3

In the container accommodation body according to the application example, the reagent may be freeze dried.

According to the container accommodation body of this application example, water content contained in the reagent for performing the nucleic acid amplification reaction is capable of being reduced, and more preferably, the reagent is capable of being prevented from containing water content.

### Application Example 4

In the container accommodation body according to the application example, the drying agent may be molecular sieve.

According to the container accommodation body of this application example, even if water enters the package, since the drying agent absorbs the water, the water is capable of being restricted from coming into contact with the reagent.

### Application Example 5

In the container accommodation body according to the application example, the container may accommodate and seal a fluid that is not mixed with the reagent.

According to the container accommodation body of this application example, the reagent and the fluid are capable of being restricted from being mixed together.

### Application Example 6

In the container accommodation body according to the application example, the reagent may be disposed in the fluid, and the fluid may be dehydrated.

According to the container accommodation body of this application example, the fluid is capable of being prevented from containing water.

### Application Example 7

In the container accommodation body according to the application example, the package may be a bag with an aluminum layer.

According to the container accommodation body of this application example, the aluminum layer is capable of decreasing the water permeability of the package.

### BRIEF DESCRIPTION OF THE DRAWINGS

The invention will be described with reference to the accompanying drawings, wherein like numbers reference like elements.

FIG. 1 is a front view of a container assembly in an embodiment.

FIG. 2 is a side view of the container assembly in the embodiment.

FIG. 3 is a plan view of the container assembly in the embodiment.

FIG. 4 is a perspective view of the container assembly in the embodiment.

FIG. 5 is a sectional view of the container assembly taken along line A-A in FIG. 3 in the embodiment.

FIG. 6 is a sectional view of the container assembly taken along line C-C in FIG. 3 in the embodiment.

FIGS. 7A and 7B are schematic views illustrating the operation of the container assembly in the embodiment.

FIGS. 8A and 8B are schematic views illustrating the operation of the container assembly in the embodiment.

FIG. 9 is a view illustrating a schematic configuration of a PCR apparatus.

FIG. 10 is a block diagram of the PCR apparatus.

FIG. 11 illustrates sectional views of a cartridge set in the embodiment.

FIG. 12 is a sectional view of a first package in the embodiment.

FIG. 13 is a sectional view of a first temporary assembly in the embodiment.

FIG. 14 is a sectional view of a second temporary assembly in the embodiment.

FIG. 15 is a sectional view of a reaction container in the embodiment.

FIG. 16 illustrates sectional views of the cartridge set in the embodiment.

FIG. 17 illustrates sectional views of a cartridge set in a modification example of the embodiment.

### DESCRIPTION OF EXEMPLARY EMBODIMENTS

Hereinafter, a preferred embodiment of the invention will be described in detail with reference to the accompanying drawings. The embodiment to be described hereinbelow does not improperly limit the scope of the invention which is stated in the appended claims. All of configuration elements to be described hereinbelow are not necessarily essential configuration elements of the invention.

A cartridge set according to the invention is sets which are used to assemble a cartridge for performing PCR. That is, it is possible to obtain a cartridge for performing PCR by assembling a cartridge set according to the invention. Hereinafter, first, the cartridge (container assembly) will be described, and then the cartridge set will be described.

### 1. Outline of Container Assembly

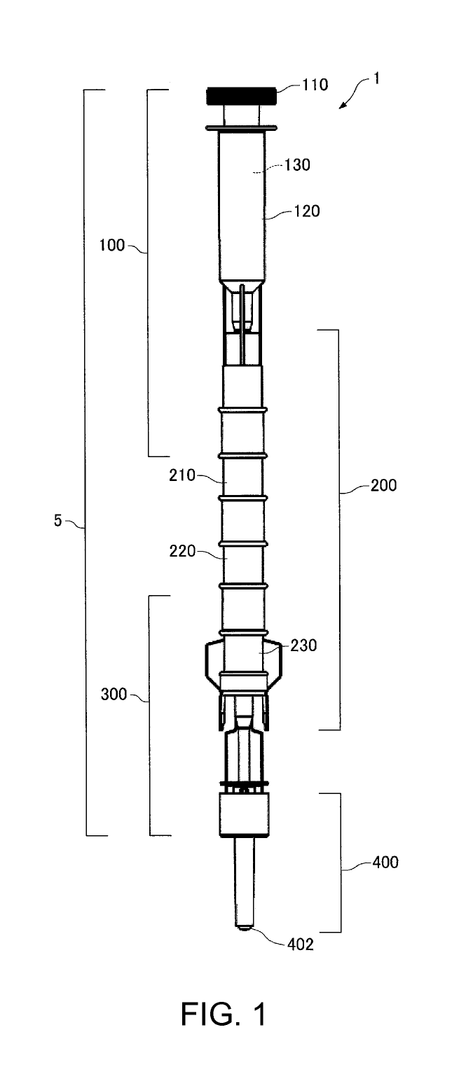

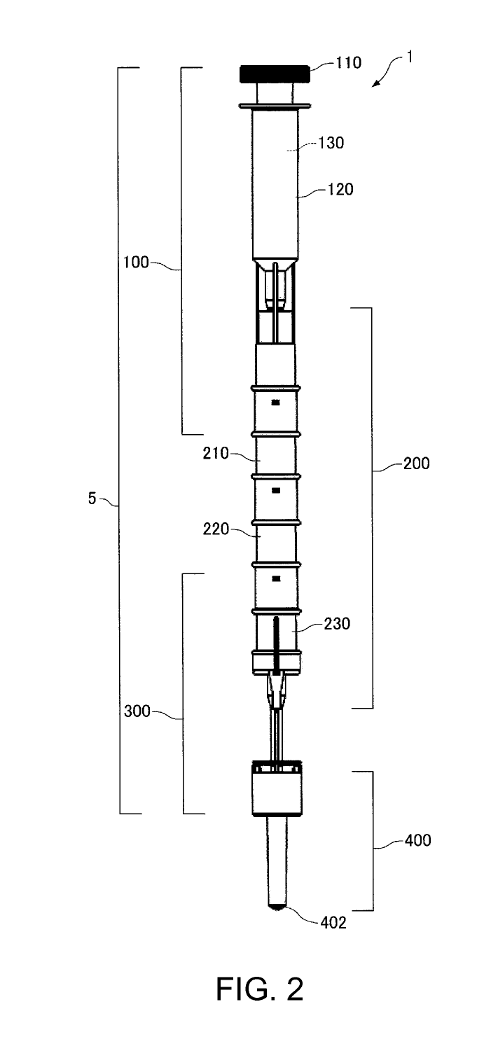

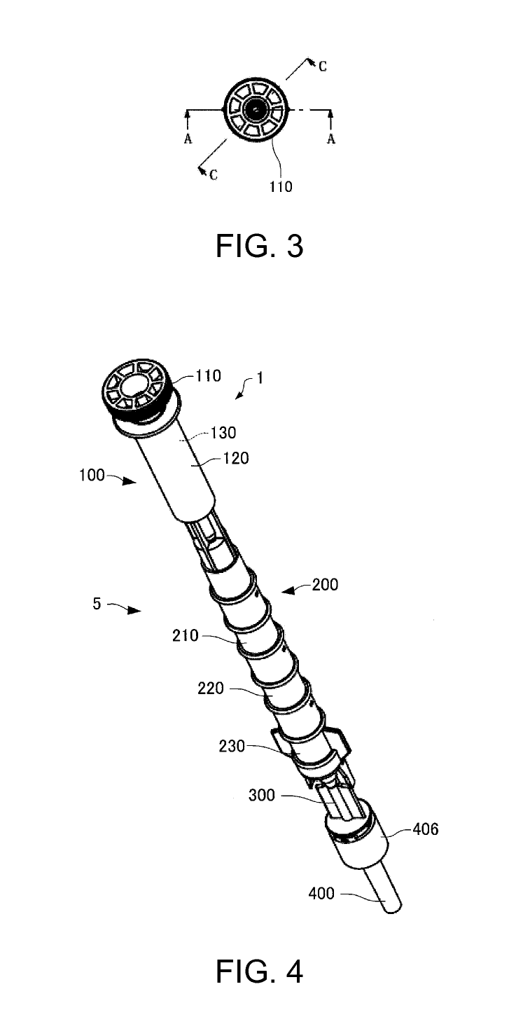

First, an outline of a container assembly 1 in the embodiment will be described with reference to FIGS. 1 to 4. FIG. 1 is a front view of the container assembly (hereinafter, which may be referred to as a cartridge) 1 in the embodiment. FIG. 2 is a side view of the container assembly 1 in the embodiment. FIG. 3 is a plan view of the container assembly 1 in the embodiment. FIG. 4 is a perspective view of the container assembly 1 in the embodiment. The description will be given with the container assembly 1 in FIGS. 1 to 3 standing upright.

The container assembly 1 includes an adsorption container 100; a cleaning container 200; an elution container 300; and a reaction container 400. The container assembly 1 is a container in which a flow passage (not illustrated) is provided from the adsorption container 100 up to the reaction container 400. One end portion of the flow passage of the container assembly 1 is closed with a cap 110, and the other end portion is closed with a bottom portion 402.

The container assembly 1 is a container in which a pre-process and a thermal cycling process are performed. In the pre-process, nucleic acid is bound with a magnetic bead (not illustrated) in the adsorption container 100, the magnetic bead is purified while moving in the cleaning container 200, and the nucleic acid is eluted into an elution solution droplet (not illustrated) in the elution container 300, and in the thermal cycling process, the elution solution droplet containing the nucleic acid undergoes a polymerase reaction in the reaction container 400.

The material of the container assembly 1 is not limited to a specific material, and glass, a polymer, metal, or the like may be used as the material of the container assembly 1. Glass, a polymer, or the like having transparency for visible light is preferably selected as the material of the container assembly 1 because the interior (cavity) of the container assembly 1 is capable of being observed from the outside of the container assembly 1. A substance transparent to a magnetic force or a non-magnetic material is preferably selected as the material of the container assembly 1 because the magnetic bead (not illustrated) is allowed to easily pass through the container assembly 1 by applying a magnetic force to the magnetic bead from the outside of the container assembly 1. For example, the material of the container assembly 1 may be polypropylene resin.

The adsorption container 100 includes a cylindrical syringe portion 120 that accommodates an adsorption solution (not illustrated); a plunger portion 130 that is a movable plunger inserted into the syringe portion 120; and the cap 110 that is fixed to one end portion of the plunger portion 130. When the cap 110 is moved relative to the syringe portion 120 in the adsorption container 100, the plunger portion 130 slides against an inner surface of the syringe portion 120, and the adsorption solution (not illustrated) accommodated in the syringe portion 120 is capable of being pushed out into the cleaning container 200. The adsorption solution will be described later.

The cleaning container 200 is obtained by assembling together a first cleaning container 210, a second cleaning container 220, and a third cleaning container 230 using a joining method. Each of the first cleaning container 210, the second cleaning container 220, and the third cleaning container 230 has one or more cleaning solution layers therein, with the cleaning solution layers being divided off by an oil layer (not illustrated). The first cleaning container 210, the second cleaning container 220, and the third cleaning container 230 are joined together such that the cleaning container 200 has multiple cleaning solution layers therein, with the multiple cleaning solution layers being divided off by multiple oil layers (not illustrated). In the example given in the embodiment, the cleaning container 200 includes three cleaning containers, that is, the first cleaning container 210, the second cleaning container 220, and the third cleaning container 230; however, the number of cleaning containers used is not limited to three, and may be increased or decreased according to the number of cleaning solution layers. The cleaning solution will be described later.

The elution container 300 is joined with the third cleaning container 230 of the cleaning container 200, and accommodates the elution solution in such a way that the shape of a plug of the elution solution is capable of being maintained. The term “plug” referred to here represents a specific liquid that occupies a section in a flow passage. More specifically, a plug of a specific liquid has the shape of a column that is occupied by substantially only the specific liquid in the flow passage in a longitudinal direction of the flow passage, and a predetermined space in the flow passage is divided off by the liquid plug. The term “substantially” referred to here implies that a small amount of (for example, thin film-like) other substances (liquid and the like) are present around the plug, that is, on an internal wall of the flow passage. The elution solution will be described later.

A nucleic acid purification device 5 includes the adsorption container 100; the cleaning container 200; and the elution container 300.

The reaction container 400 is joined with the elution container 300, and is a container that receives the liquid that is pushed out of the elution container 300, and accommodates the elution solution droplet containing the specimen in the thermal cycling process. The reaction container 400 accommodates a reagent (not illustrated). The reagent will be described later.

### 2. Detailed Structure of Container Assembly

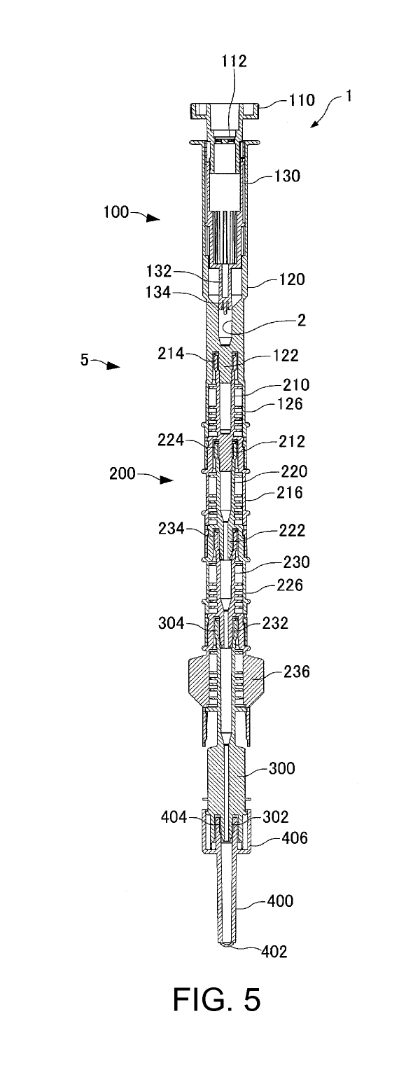

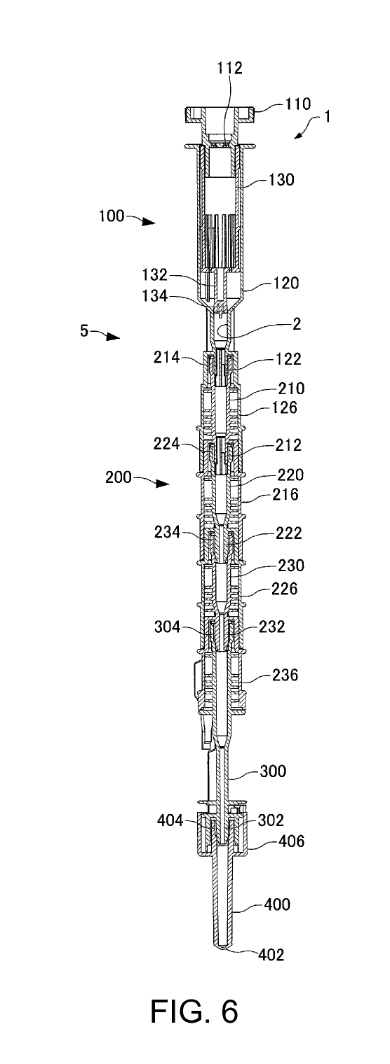

Hereinafter, a detailed structure of the container assembly 1 will be described with reference to FIGS. 5 and 6. FIG. 5 is a sectional view of the container assembly 1 taken along line A-A in FIG. 3 in the embodiment. FIG. 6 is a sectional view of the container assembly 1 taken along line C-C in FIG. 3 in the embodiment. Actually, the container assembly 1 is assembled in a state where the container assembly 1 is filled with contents such as the cleaning solution; however, in FIGS. 5 and 6, the contents are not illustrated for the purpose of illustrating the structure of the container assembly 1.

### 2-1. Adsorption Container

In the adsorption container 100, the plunger portion 130 is inserted into one open end portion of the syringe portion 120, and the cap 110 is inserted into an open end portion of the plunger portion 130. The cap 110 has an air ventilation portion 112 at the center thereof, and the air ventilation portion 112 is capable of restricting a change in the internal pressure of the plunger portion 130 that is in operation.

The plunger portion 130 is a substantially cylindrical plunger that slides against an inner circumferential surface of the syringe portion 120. The plunger portion 130 includes the open end portion into which the cap 110 is inserted; a bar-shaped portion 132 that extends in a longitudinal direction of the syringe portion 120 from a bottom portion facing the open end portion; and a tip end portion 134 at a tip end of the bar-shaped portion 132. The bar-shaped portion 132 protrudes from the center of a bottom portion of the plunger portion 130. Through holes are provided in the circumference of the bar-shaped portion 132, and the interior of the plunger portion 130 communicates with the interior of the syringe portion 120.

The syringe portion 120 forms a portion of a flow passage 2 of the container assembly 1, and includes a large-diameter portion that accommodates the plunger portion 130; a small-diameter portion that has an inner diameter smaller than that of the large-diameter portion; a decreasing diameter portion that has an inner diameter decreasing from the inner diameter of the large-diameter portion to the inner diameter of the small-diameter portion; an adsorption insertion portion 122 at a tip end of the small-diameter portion; and a cylindrical adsorption cover portion 126 that covers the circumference of the adsorption insertion portion 122. Each of the large-diameter portion, the small-diameter portion, and the adsorption insertion portion 122 forms a portion of the flow passage 2 of the container assembly 1, and has a substantially cylindrical shape.

When the container assembly 1 is supplied to an operator, the tip end portion 134 of the plunger portion 130 seals the small-diameter portion of the syringe portion 120, and divides the plunger portion 130 into a section of the large-diameter portion and another section of the decreasing diameter portion such that two sections are formed.

The adsorption insertion portion 122 of the syringe portion 120 is inserted and fitted into a first receiving portion 214 that is one open end portion of the first cleaning container 210 of the cleaning container 200 such that the syringe portion 120 is joined with the first cleaning container 210. An outer circumferential surface of the adsorption insertion portion 122 is in close contact with an inner circumferential surface of the first receiving portion 214 such that the liquid (the contents) is prevented from leaking to the outside.

### 2-2. Cleaning Container

The cleaning container 200 forms a portion of the flow passage 2 of the container assembly 1, and is an assembly of the first cleaning container 210, the second cleaning container 220, and the third cleaning container 230. Since the first cleaning container 210, the second cleaning container 220, and the third cleaning container 230 have the same basic structure, only the structure of the first cleaning container 210 will be described, and descriptions of the second cleaning container 220 and the third cleaning container 230 will be omitted.

The first cleaning container 210 has a substantially cylindrical shape extending in a longitudinal direction of the container assembly 1. The first cleaning container 210 includes a first insertion portion 212 that is provided in the other open end portion of the first cleaning container 210; the first receiving portion 214 that is provided in the one open end portion thereof; and a first cover portion 216 with a cylindrical shape which covers the circumference of the first insertion portion 212.

The first insertion portion 212 has an outer diameter that is substantially the same as the inner diameter of a second receiving portion 224. The first receiving portion 214 has an inner diameter that is substantially the same as the outer diameter of the adsorption insertion portion 122.

The first insertion portion 212 of the first cleaning container 210 is inserted and fitted into the second receiving portion 224 of the second cleaning container 220, and thus an outer circumference of the first insertion portion 212 is in close contact with an inner circumference of the second receiving portion 224 such that the gap therebetween is sealed, and the first cleaning container 210 is joined with the second cleaning container 220. Similarly, the first cleaning container 210, the second cleaning container 220, and the third cleaning container 230 are connected to each other such that the cleaning container 200 is formed. The term “sealing” referred to here implies that a container is sealed in such a way that at least a liquid or gas accommodated therein is prevented from leaking to the outside, and may also imply that the container is sealed in such a way that a liquid or gas is prevented from infiltrating thereinto from the outside.

### 2-3. Elution Container

The elution container 300 has a substantially cylindrical shape extending in the longitudinal direction of the container assembly 1, and forms a portion of the flow passage 2 of the container assembly 1. The elution container 300 includes an elution insertion portion 302 that is provided in one open end portion of the elution container 300, and an elution receiving portion 304 that is provided in the other open end portion thereof.

The elution receiving portion 304 has an inner diameter that is substantially the same as the outer diameter of a third insertion portion 232 of the third cleaning container 230. The third insertion portion 232 is inserted and fitted into the elution receiving portion 304, and thus an outer circumference of the third insertion portion 232 is in close contact with an inner circumference of the elution receiving portion 304 such that the gap therebetween is sealed, and the third cleaning container 230 is joined with the elution container 300.

### 2-4. Reaction Container

The reaction container 400 has a substantially cylindrical shape extending in the longitudinal direction of the container assembly 1, and forms a portion of the flow passage 2 of the container assembly 1. The reaction container 400 includes a reaction receiving portion 404 that is provided in one open end portion of the reaction container 400; the bottom portion 402 that is provided in the other closed end portion thereof; and a reservoir portion 406 that covers the reaction receiving portion 404.

The reaction receiving portion 404 has an inner diameter that is substantially the same as the outer diameter of the elution insertion portion 302 of the elution container 300. The elution insertion portion 302 is inserted and fitted into the reaction receiving portion 404 such that the elution container 300 is joined with the reaction container 400.

The reservoir portion 406 is provided on the circumference of the reaction receiving portion 404, and has a predetermined space. The reservoir portion 406 has a volume such that the reservoir portion 406 is capable of accommodating the liquid which overflows from the reaction container 400 due to the movement of the plunger portion 130.

### 3. Contents of Container Assembly and Operation of Container Assembly

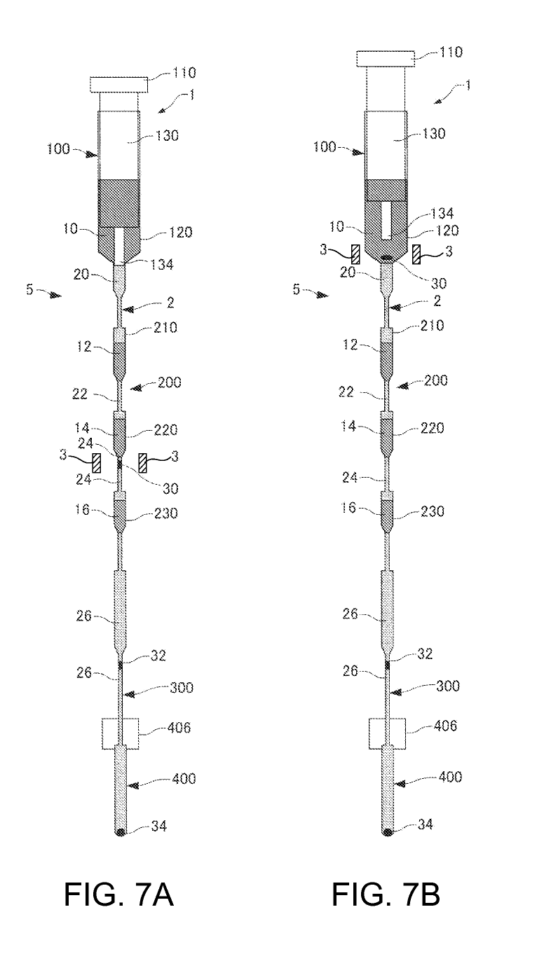

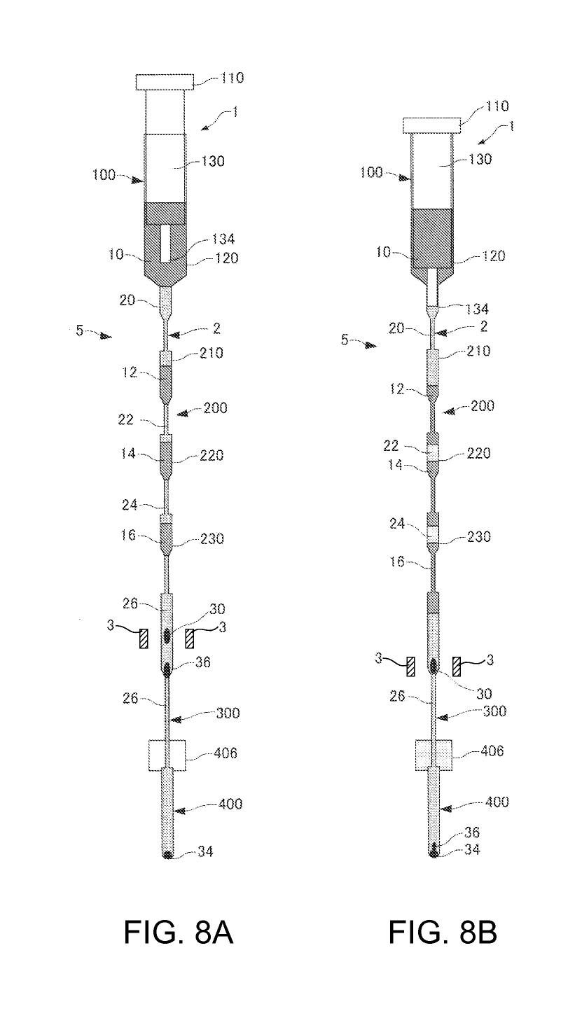

Hereinafter, the contents of the container assembly 1 will be described with reference to FIG. 7A, and the operation of the container assembly 1 will be described with reference to FIGS. 7A, 7B, 8A, and 8B. FIGS. 7A and 7B are schematic views illustrating the operation of the container assembly 1 in the embodiment. FIGS. 8A and 8B are schematic views illustrating the operation of the container assembly 1 in the embodiment. In FIGS. 7A, 7B, 8A, and 8B, for the purpose of illustrating states of the contents, each of the containers is illustrated by the flow passage 2, and the exteriors or the joining structures of the containers are not illustrated.

### 3-1. Contents

FIG. 7A illustrates the states of the contents in the flow passage 2 when the container assembly 1 is in the state illustrated in FIG. 1. The flow passage 2 contains the following contents which are sequentially disposed from the cap 110 to the reaction container 400: an adsorption solution 10; a first oil 20; a first cleaning solution 12; a second oil 22; a second cleaning solution 14; a third oil 24; a magnetic bead 30; the third oil 24; a third cleaning solution 16; a fourth oil 26; an elution solution 32; the fourth oil 26; and a reagent 34.

The flow passage 2 has portions with large sectional areas (wide portions of the flow passage 2) and portions with small sectional areas (narrow portions of the flow passage 2) which are alternatingly disposed, with each of the sectional areas being an area of a surface perpendicular to the longitudinal direction of the container assembly 1. A portion or the entirety of each of the first oil 20, the second oil 22, the third oil 24, the fourth oil 26, and the elution solution 32 is accommodated in the narrow portion of the flow passage 2. The narrow portion of the flow passage 2 has an area in which the boundary surface is capable of being stably maintained when the boundary surface between adjacent and non-mixable liquids (which may be fluids hereinafter) is disposed in the narrow portion of the flow passage 2. Accordingly, a liquid disposed in the narrow portion of the flow passage 2 is capable of stably maintaining a dispositional relationship between the liquid and other liquids which are disposed above and below the liquid. Also in a case where the boundary surface between a liquid disposed in the narrow portion of the flow passage 2 and another liquid disposed in the wide portion of the flow passage 2 is disposed in the wide portion of the flow passage 2, even if the boundary surface is disturbed by a strong impact, the placement of the container assembly 1 in a stationary state stabilizes the boundary surface at a predetermined position.

The narrow portions of the flow passage 2 are respectively formed inside the adsorption insertion portion 122; the first insertion portion 212, the second insertion portion 222, the third insertion portion 232, and the elution insertion portion 302, and the narrow portion of the flow passage 2 extends upwards exceeding the elution insertion portion 302 of the elution container 300. The liquids accommodated in the narrow portions of the flow passage 2 are stably held even before the containers are assembled together.

### 3-1-1. Oil

Each of the first oil 20, the second oil 22, the third oil 24, and the fourth oil 26 is an oil, and is present as a plug between a liquid therebefore and a liquid thereafter in the state illustrated in FIGS. 7A and 7B. Since each of the first oil 20, the second oil 22, the third oil 24, and the fourth oil 26 is present as a plug, phase-separable liquids, that is, non-mixable liquids are selected as liquids which are adjacent to each other with each oil interposed therebetween. The first oil 20, the second oil 22, the third oil 24, and the fourth oil 26 may be different types of oils. Any one type out of the following oils may be used as the first oil 20, the second oil 22, the third oil 24, and the fourth oil 26: silicone oils such as a dimethylsilicone oil; paraffin oils; mineral oils; and the mixtures thereof.

### 3-1-2. Adsorption Solution

The adsorption solution 10 is a liquid serving as a ground on which nucleic acid adsorbs to the magnetic bead 30, and for example, is an aqueous solution containing a chaotropic substance. 5M guanidine thiocyanate, 2% Triton X-100, 50 mM Tris-HCl (pH 7.2), or the like may be used as the adsorption solution 10. Insofar as the adsorption solution 10 contains a chaotropic substance, the adsorption solution 10 is not limited to a specific solution, and the adsorption solution 10 may contain a surfactant for the purpose of destroying a cell membrane or denaturalizing proteins contained in a cell. Insofar as the surfactant may be used to typically extract nucleic acid from a cell or the like, the surfactant is not limited to a specific type of surfactant. Specifically, anyone of the following surfactants may be used: non-ionic surfactants such as Triton series surfactants (Triton X-series surfactants and the like) and Tween surfactants (a Tween 20 surfactant and the like); anionic surfactants such as an N-lauroylsarcosine sodium (SDS); and the like, and in particular, 0.1% to 2% of a non-ionic surfactant is preferably used. In addition, the adsorption solution 10 preferably contains a reducing agent such as 2-mercaptoethanol or dithiothreitol. A solution may be a buffer solution, and preferably has a neutral pH of 6 to 8. Specifically, with all these factors taken into consideration, the adsorption solution 10 preferably contains 3 M to 7 M of guanidine salt, 0% to 5% of a non-ionic surfactant, 0 mM to 0.2 mM of EDTA, 0 M to 0.2 M of a reducing agent, and the like.

Insofar as the chaotropic substance acts to produce chaotropic ions (anions with an ion radius of univalence) in an aqueous solution, and to increase the water solubility of hydrophobic molecules, and helps nucleic acid to adsorb to a solid-phase carrier, the chaotropic substance is not limited to a specific type of chaotropic substance. Specifically, examples of the chaotropic substance include guanidine hydrochloride, sodium iodide, sodium perchlorate, and the like. Among the chaotropic substances, guanidine thiocyanate or guanidine hydrochloride, which strongly acts to denaturalize proteins, is preferably used. The concentration of the chaotropic substance is changed according to the selected substance, and for example, when guanidine thiocyanate is used as the chaotropic substance, the concentration is preferably present in a range of 3 M to 5.5 M, and when guanidine hydrochloride is used as the chaotropic substance, the concentration is preferably greater than or equal to 5 M.

Due to the presence of the chaotropic substance in the aqueous solution, it is thermodynamically advantageous for the nucleic acid in the aqueous solution to be present while adsorbing to the solid rather than being present while being surrounded by water molecules, and thus the chaotropic substance adsorbs to the surface of the magnetic bead 30.

### 3-1-3. Cleaning Solution

The first cleaning solution 12, the second cleaning solution 14, and the third cleaning solution 16 clean the magnetic bead 30 with which the nucleic acid is bound.

The first cleaning solution 12 is a liquid capable of being phase-separated from both the first oil 20 and the second oil 22. The first cleaning solution 12 preferably is water or an aqueous solution with a low salt concentration, and when the first cleaning solution 12 is an aqueous solution with a low salt concentration, the first cleaning solution 12 preferably is a buffer solution. An aqueous solution with a low salt concentration preferably has a salt concentration of less than or equal to 100 mM, preferably, less than or equal to 50 mM, and the most preferably, less than or equal to 10 mM. The first cleaning solution 12 may contain the aforementioned surfactant, and the pH of the first cleaning solution 12 is not limited to a specific pH value. A salt for making the first cleaning solution 12 of a buffer solution is not limited to a specific type of salt, and salts such as tris, hepes, pipes, and phosphoric acid are preferably used. The first cleaning solution 12 preferably contains an amount of alcohol to the extent that the alcohol does not adversely restrict the adsorption of the nucleic acid to the carrier, a reverse transcription reaction, a PCR, and the like. At this time, the concentration of the alcohol is not limited to a specific concentration.

The first cleaning solution 12 may contain the chaotropic substance. For example, when the first cleaning solution 12 contains guanidine hydrochloride, the magnetic bead 30 is capable of being cleaned while the adsorption of the nucleic acid to the magnetic bead 30 or the like is maintained or intensified.

The second cleaning solution 14 is a liquid capable of being phase-separated from both the second oil 22 and the third oil 24. Basically, the second cleaning solution 14 may have the same composition as, or a different composition from that of the first cleaning solution 12, and the second cleaning solution 14 preferably is a solution that actually does not contain the chaotropic substance. The reason for this is that the chaotropic substance is prevented from being brought into a subsequent solution. For example, the second cleaning solution 14 may be 5 mM tris-hydrochloric acid buffer solution. As described above, the second cleaning solution 14 preferably contains alcohol.

The third cleaning solution 16 is a liquid capable of being phase-separated from both the third oil 24 and the fourth oil 26. Basically, the third cleaning solution 16 may have the same composition as, or a different composition from that of the second cleaning solution 14, and does not contain alcohol. The third cleaning solution 16 may contain citric acid so that alcohol can be prevented from being brought into the reaction container 400.

### 3-1-4. Magnetic Bead

The magnetic bead 30 is a bead that adsorbs the nucleic acid, and preferably has relatively strong magnetic properties so that the magnetic bead 30 is capable of moving due to a magnet 3 outside the container assembly 1. The magnetic bead 30 may be a silica bead, or a silica-coated bead. The magnetic bead 30 preferably is a silica-coated bead.

### 3-1-5. Elution Solution

The elution solution 32 is a liquid capable of being phase-separated from the fourth oil 26, and is present as a plug in the flow passage 2 of the elution container 300 while being interposed between the fourth oils 26, 26. The elution solution 32 is a liquid that elutes the nucleic acid (which has adsorbed to the magnetic bead 30) from the magnetic bead 30 thereinto. The elution solution 32 is transformed into a droplet in the fourth oil 26 by heating. For example, pure water may be used as the elution solution 32. The term “droplet” referred to here is a liquid surrounded with a free surface.

### 3-1-6. Reagent

The reagent 34 contains components necessary for a reaction. When the PCR occurs in the reaction container 400, the reagent 34 may contain at least one of an enzyme (DNA polymerase or the like) and a primer (nucleic acid) for amplifying target nucleic acid (DNA) eluted in a droplet 36 (refer to FIGS. 8A and 8B) of the elution solution, and a fluorescent probe for detecting an amplicon, and in the example, the reagent 34 contains all of the primer, the enzyme, and the fluorescent probe. The reagent 34 is not compatible with the fourth oil 26. When the reagent 34 comes into contact with the droplet 36 of the elution solution 32, with the droplet 36 containing the nucleic acid, the reagent 34 dissolves and undergoes a reaction. The reagent 34 is present in a solid state in the lowest region of the flow passage 2 of the reaction container 400 in the direction of gravity. For example, a freeze-dried reagent may be used as the reagent 34.

### 3-2. Operation of Container Assembly

An example of the operation of the container assembly 1 will be described with reference to FIGS. 7A, 7B, 8A, and 8B.

The operation of the container assembly 1 includes (A) a step of assembling the container assembly 1 by joining together the adsorption container 100, the cleaning container 200, the elution container 300, and the reaction container 400; (B) a step of introducing a specimen containing nucleic acid into the adsorption container 100 that accommodates the adsorption solution 10; (C) a step of moving the magnetic bead 30 from the second cleaning container 220 to the adsorption container 100; (D) a step of causing the nucleic acid to adsorb to the magnetic bead 30 by shaking the adsorption container 100, (E) a step of moving the magnetic bead 30 (to which the nucleic acid adsorbs) from the adsorption container 100 to the elution container 300, while causing the magnetic bead 30 to sequentially pass through the first oil 20, the first cleaning solution 12, the second oil 22, the second cleaning solution 14, the third oil 24, the third cleaning solution 16, and the fourth oil 26; (F) a step of eluting the nucleic acid from the magnetic bead 30 into the elution solution 32 in the elution container 300; and (G) a step of bringing a droplet containing the nucleic acid into contact with the reagent 34 in the reaction container 400.

Hereinafter, each of the steps will be sequentially described.

### (A) Step of Assembling Container Assembly 1

As illustrated in FIG. 7A, in the assembly step, the container assembly 1 is assembled in such a way that the adsorption container 100 to the reaction container 400 are joined together, and the flow passage 2 is continuously formed from the adsorption container 100 to the reaction container 400. In FIG. 7A, the cap 110 is mounted on the adsorption container 100; however, after step (B), the cap 110 is mounted on the plunger portion 130.

More specifically, the elution insertion portion 302 of the elution container 300 is inserted into the reaction receiving portion 404 of the reaction container 400, the third insertion portion 232 of the third cleaning container 230 is inserted into the elution receiving portion 304 of the elution container 300, the second insertion portion 222 of the second cleaning container 220 is inserted into the third receiving portion 234 of the third cleaning container 230, the first insertion portion 212 of the first cleaning container 210 is inserted into the second receiving portion 224 of the second cleaning container 220, and the adsorption insertion portion 122 of the adsorption container 100 is inserted into the first receiving portion 214 of the first cleaning container 210.

### (B) Step of Introducing Specimen

The introduction step is performed in such a way that a cotton swab to which a specimen adheres is inserted into the adsorption solution 10 via an opening of the adsorption container 100, on which the cap 110 is mounted, and the cotton swab is immersed in the adsorption solution 10. More specifically, the cotton swab is inserted into the opening in one end portion of the plunger portion 130 that is inserted into the syringe portion 120 of the adsorption container 100. Subsequently, the cotton swab is taken out of the adsorption container 100, and the cap 110 is mounted on the adsorption container 100. The container assembly 1 in this state is illustrated in FIG. 7A. In addition, the specimen may be introduced into the adsorption container 100 by means of a pipette or the like. When the specimen is in a paste state or a solid state, the specimen may be adhered to an internal wall of the plunger portion 130 or put into the adsorption container 100 by means of a spoon, forceps, or the like. As illustrated in FIG. 7A, the syringe portion 120 and the plunger portion 130 are filled with the adsorption solution 10 while the adsorption solution 10 is filled up to a midpoint thereof, and a space is present on an opening side of the plunger portion 130 on which the cap 110 is mounted.

The specimen contains the nucleic acid which is a target. Hereinafter, the specimen containing the nucleic acid may be simply referred to as target nucleic acid. For example, the target nucleic acid is deoxyribonucleic acid (DNA) and/or ribonucleic acid (RNA). After the target nucleic acid is extracted from the specimen, and is eluted into the elution solution 32 (to be described), the target nucleic acid is used as a mold of the PCR. Examples of the specimens include blood, nasal mucus, an oral mucous membrane, and other various biological specimens.

### (C) Step of Moving Magnetic Bead

The step of moving the magnetic bead 30 is performed by moving the magnet 3 toward the adsorption container 100 while a magnetic force of the magnet 3 disposed outside the containers is applied to the magnetic bead 30 that is present in the shape of a plug while being interposed between the third oils 24, 24 of the second cleaning container 220 as illustrated in FIG. 7A.

Following or prior to the movement of the magnetic bead 30, the cap 110 and the plunger portion 130 are moved in a direction in which the cap 110 and the plunger 130 are pulled out of the syringe portion 120, and thus the specimen in the adsorption solution 10 moves from the plunger portion 130 into the syringe portion 120. Due to the movement of the plunger portion 130, the flow passage 2 blocked by the tip end portion 134 communicates with the adsorption solution 10.

The magnetic bead 30 moves upwards in the flow passage 2 along with the movement of the magnet 3, and as illustrated in FIG. 7B, the magnetic bead 3 reaches the adsorption solution 10 in which the specimen is present.

### (D) Step of Causing Nucleic Acid to Adsorb to Magnetic Bead

The step of causing the nucleic acid to adsorb to the magnetic bead 30 is performed by shaking the adsorption container 100. Since an opening of the adsorption container 100 is sealed with the cap 110 so as to prevent a leak of the adsorption solution 10, the step may be efficiently performed. In the step, the target nucleic acid adsorbs to the surface of the magnetic bead 30 due to an action of a chaotropic agent. In the step, nucleic acid or proteins other than the target nucleic acid may adsorb to the surface of the magnet bead 30.

The adsorption container 100 may be shaken using a device such as a well-known vortex shaker, or with the hands of an operator. The adsorption container 100 may be shaken while a magnetic field induced by the magnetic properties of the magnetic bead 30 is applied to the adsorption container 100 from the outside.

(E) Step of Moving Magnetic Bead to which Nucleic Acid Adsorbs

In the step of moving the magnetic bead 30 to which the nucleic acid adsorbs, the magnet 3 is moved while applying a magnetic force from the outside of the adsorption container 100, the cleaning container 200, and the elution container 300, and thus the magnetic bead 30 moves through the adsorption solution 10, the first oil 20, the second oil 22, the third oil 24, the fourth oil 26, the first cleaning solution 12, the second cleaning solution 14, and the third cleaning solution 16.

The magnet 3 may be a permanent magnet, an electromagnet, or the like. The magnet 3 may be moved with the hands of an operator, or using a mechanical device or the like. The magnetic bead 30 has properties of gravitating to the magnet 3 due to the magnetic force of the magnet 3, and the disposition of the magnet 3 relative to the adsorption container 100, the cleaning container 200, and the elution container 300 is changed, and the magnetic bead 30 is moved in the flow passage 2 using these properties. The speed of the magnetic bead 30 passing through each of the cleaning solutions is not limited to a specific speed, and the magnetic bead 30 may move while reciprocating in the same cleaning solution along the longitudinal direction of the flow passage 2. Particles and the like other than the magnetic bead 30 are capable of moving in the tubes due to gravity, an electric potential difference, or the like.

### (F) Step of Eluting Nucleic Acid

In the step of eluting the nucleic acid, the nucleic acid is eluted from the magnet bead 30 into the droplet 36 of the elution solution in the elution container 300. The elution solution 32 illustrated in FIGS. 7A and 7B is present as a plug in the narrow portion of the flow passage of the elution container 300. The reaction container 400 is heated while the magnetic bead 30 moves as described above, and thus the content fluid expands, and as illustrated in FIGS. 8A and 8B, the elution solution 32 moves upwards in the elution container 300 in the form of the droplet 36. As illustrated in FIG. 8A, when the magnetic bead 30 reaches the droplet 36 of the elution solution in the elution container 300, due to an action of the elution solution, the target nucleic acid adsorbing to the magnetic bead 30 is eluted into the droplet 36 of the elution solution.

(G) Step of Bringing Droplet 36 into Contact with Reagent 34

In the step of bringing the droplet 36 into contact with the reagent 34, the droplet 36 containing the nucleic acid is brought into contact with the reagent 34 that is present at the lowest region in the reaction container 400. Specifically, as illustrated in FIG. 8B, the cap 110 is pressed, and the first oil 20 is pressed downwards by the tip end portion 134 of the plunger portion 130, and thus, in a state where the magnetic force of the magnet 3 is applied to the magnetic bead 30, and the magnetic bead 30 is held at a predetermined position, the droplet 36 of the elution solution, to which the target nucleic acid is eluted, moves toward the reaction container 400, and comes into contact with the reagent 34 that is present at the lowest region of the reaction container 400. The reagent 34 in contact with the droplet 36 is dissolved, and is mixed with the target nucleic acid in the elution solution, and the PCR using thermal cycling is capable of being realized.

### 4. PCR Apparatus

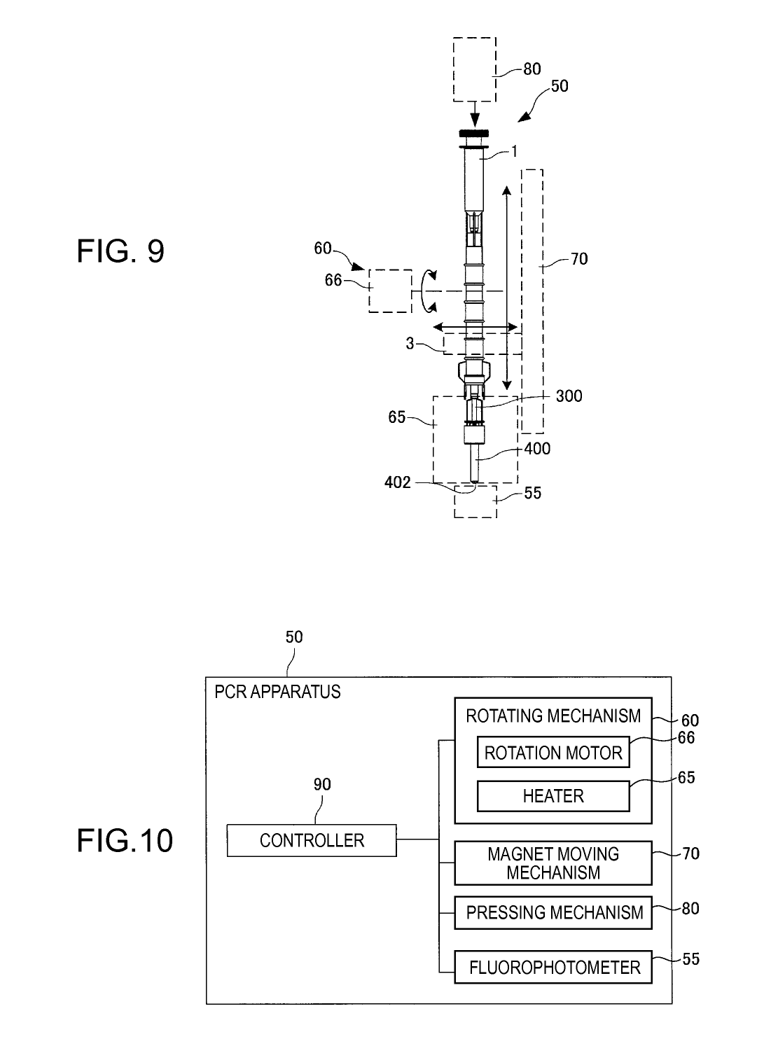

A PCR apparatus 50, which performs a nucleic acid elution process and the PCR using the container assembly 1, will be described with reference to FIGS. 9 and 10. FIG. 9 is a view illustrating a schematic configuration of the PCR apparatus 50. FIG. 10 is a block diagram of the PCR apparatus 50.

The PCR apparatus 50 includes a rotating mechanism 60; a magnet moving mechanism 70; a pressing mechanism 80; a fluorophotometer 55; and a controller 90.

### 4-1. Rotating Mechanism

The rotating mechanism 60 includes a rotation motor 66 and a heater 65, and the rotation motor 66 is driven such that the container assembly 1 and the heater 65 are rotated. The rotating mechanism 60 rotates the container assembly 1 and the heater 65 such that the container assembly 1 and the heater 65 are inverted upside down, and thus, the droplet containing the target nucleic acid moves in the flow passage of the reaction container 400, and the thermal cycling is performed.

The heater 65 includes multiple heaters (not illustrated), and may include an elution heater, a high-temperature heater, a low-temperature heater, and the like. The elution heater promotes the elution of the target nucleic acid from the magnetic bead into the elution solution by heating the plug-shaped elution solution of the container assembly 1. The high-temperature heater heats the liquid on an upstream side of the flow passage of the reaction container 400 to a temperature that is higher than that of the low-temperature heater. The low-temperature heater heats the bottom portion 402 of the flow passage of the reaction container. The temperature gradient of the liquid in the flow passage of the reaction container 400 may be produced by the high-temperature heater and the low-temperature heater. The heater 65 is provided with a temperature control device, and the temperature control device is capable of setting the temperatures of the liquids in the container assembly 1 to be appropriate for the process according to a command from the controller 90.

The heater 65 has an opening through which an external wall of the bottom portion 402 of the reaction container 400 is exposed. The fluorophotometer 55 measures the luminance of the droplet of the elution solution via this opening.

### 4-2. Magnet Moving Mechanism

The magnet moving mechanism 70 is a mechanism that moves the magnet 3. The magnet moving mechanism 70 moves the magnetic bead in the container assembly 1 by causing the magnetic bead in the container assembly 1 to gravitate to the magnet 3, and moving the magnet 3. The magnet moving mechanism 70 includes a pair of magnets 3; a lifting up and down mechanism; and a shaking mechanism.

The shaking mechanism is a mechanism that shakes the pair of magnets 3 in a rightward and leftward direction in FIG. 9 (which may be a forward and rearward direction in FIG. 9). The pair of magnets 3 is disposed in such a way that the container assembly 1 mounted on the PCR apparatus 50 is interposed between the pair of magnets 3 in the rightward and leftward direction (refer to FIGS. 7A, 7B, 8A, and 8B), and the pair of magnets 3 is capable of reducing the distance between the magnetic bead and the magnet 3 in a direction (the rightward and leftward direction in FIG. 9) perpendicular to the flow passage of the container assembly 1. Accordingly, when the pair of magnets is shaken in the rightward and leftward direction as illustrated by the arrow, the magnetic bead in the container assembly 1 moves in the rightward and leftward direction following the movement of the magnets 3. The lifting up and down mechanism moves the magnets 3 in a vertical direction, and thus is capable of moving the magnetic bead in the vertical direction in FIG. 9 following the movement of the magnets 3.

### 4-3. Pressing Mechanism

The pressing mechanism 80 is a mechanism that presses the plunger portion of the container assembly 1. The plunger portion is pressed by the pressing mechanism 80, and thus the droplet in the elution container 300 is pushed out into the reaction container 400, and the PCR is capable of being performed in the reaction container 400.

In FIG. 9, the pressing mechanism 80 is disposed above the container assembly 1 standing upright, and the pressing mechanism 80 may press the plunger portion in the vertical direction in FIG. 9, or for example, at an angle of 45 degrees relative to the vertical direction. When the pressing mechanism 80 is disposed for a press direction to be set to an angle of 45 degrees, the pressing mechanism 80 is unlikely to interfere with the magnet moving mechanism 70.

### 4-4. Fluorophotometer

The fluorophotometer 55 measures the luminance of the droplet in the reaction container 400. The fluorophotometer 55 is disposed to face the bottom portion 402 of the reaction container 400. The fluorophotometer 55 is desirably capable of detecting the luminances of multiple wavelength bands so as to be capable of coping with multiplex PCR.

### 4-5. Controller

The controller 90 is a control unit that controls the PCR apparatus 50. The controller 90 includes, a processor such as a CPU; storage units such as a ROM and a RAM; and the like. The storage unit stores various programs and various items of data. The storage unit provides a region in which the program is deployed. The processor realizes various processes by executing the programs stored in the storage unit.

For example, the controller 90 controls the rotation motor 66 such that the container assembly 1 is rotated to a predetermined rotation position. The rotating mechanism 60 is provided with a rotation position sensor (not illustrated), and the controller 90 drives and stops the rotation motor 66 according to a detection result of the rotation position sensor.

The controller 90 controls the turning on and off of the heater 65 such that the heater 65 produces heat and the liquid inside the container assembly 1 is heated up to a predetermined temperature.

The controller 90 controls the magnet moving mechanism 70 such that the magnets 3 are moved in the vertical direction, and are shaken in the rightward and leftward direction in FIG. 9 according to a detection result of a position sensor (not illustrated).

The controller 90 controls the fluorophotometer 55 such that the fluorophotometer 55 measures the luminance of the droplet inside the reaction container 400. The result of measurement is kept in the storage unit (not illustrated) of the controller 90.

It is possible to mount the container assembly 1 on the PCR apparatus 50, and perform steps (C) to (G) described in the section 3-2, and to perform the PCR.

### 5. Cartridge Set

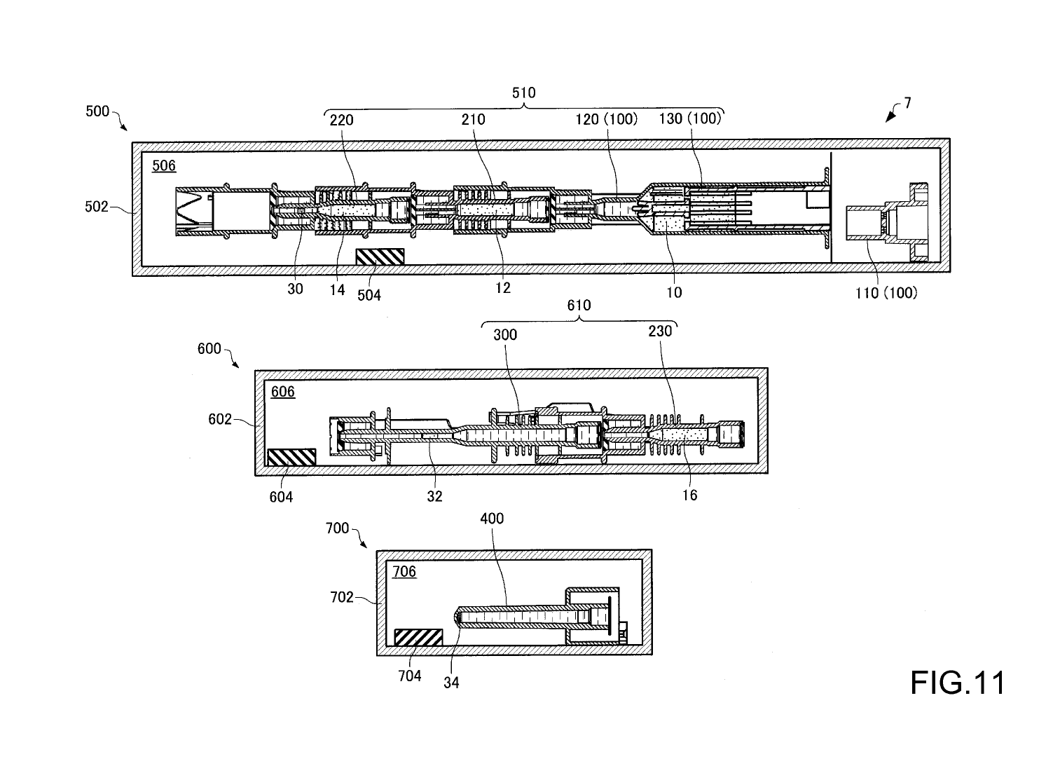

The cartridge set in the embodiment will be described with reference to the drawings. FIG. 11 shows sectional views schematically illustrating a cartridge set 7 in the embodiment. It is possible to obtain the cartridge (the container assembly) 1 by assembling the cartridge set 7.

As illustrated in FIG. 11, the cartridge set 7 includes a first accommodation body 500; a second accommodation body 600; and a third accommodation body 700. Hereinafter, the accommodation bodies 500, 600, and 700 will be described.

### 5-1. First Accommodation Body

As illustrated in FIG. 11, the first accommodation body 500 includes a first package 502; a liquid holding material 504; the syringe portion 120 and the plunger portion 130 of the adsorption container 100; the first cleaning container 210; and the second cleaning container 220. In the illustrated example, the first accommodation body 500 contains the cap 110 of the adsorption container 100.

The first package 502 accommodates and seals (tightly seals) the liquid holing material 504, the adsorption container 100, and the cleaning containers 210 and 220. In the illustrated example, the first package 502 is a bag-like package, and the shape of the first package 502 is not limited to a specific shape, and for example, may be a box shape. Insofar as the first package 502 is capable of accommodating and sealing the liquid holing material 504, the adsorption container 100, and the cleaning containers 210 and 220, the size of the first package 502 is not limited to a specific size.

The first package 502 has water permeability that is lower than that of the adsorption container 100 and the cleaning containers 210 and 220. The term “water permeability” referred to here represents the amount of water (for example, water vapor) that passes through a unit area of a package (passes through a package from the inside of the package to the outside, or from the outside of the package to the inside) per unit time at a predetermined temperature and humidity. More specifically, the term “water permeability” is water vapor permeability, and water permeability may be obtained according to JIS K7129.

The first package 502 has alcohol permeability that is lower than that of the adsorption container 100 and the cleaning containers 210 and 220. The term “alcohol permeability” referred to here represents the permeability of a package with respect to alcohol, and the amount of alcohol (for example, gaseous alcohol) that passes through a unit area of the package per unit time at a predetermined temperature and humidity. For example, the phrase “the alcohol permeability is low” may be rewritten to the phrase “gas barrier is high”. The temperature at which the water permeability and the alcohol permeability are obtained is not limited to a specific temperature, and for example, is greater than or equal to 0° C. and less than or equal to 60° C., and preferably is a room temperature. The water permeability and the alcohol permeability may be obtained according to the thickness of a package.

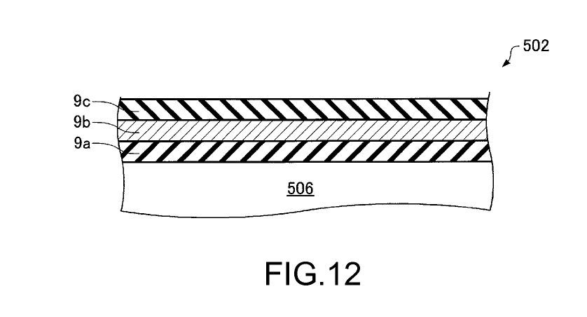

The first package 502 is preferably made of a material that has a low water vapor permeability and a high gas barrier. Specifically, the first package 502 is a bag with having an aluminum layer. FIG. 12 is a sectional view schematically illustrating the first package 502. As illustrated in FIG. 12, for example, the first package 502 includes a polypropylene (PP) layer 9a; an aluminum layer 9b that is provided on a surface of the PP layer 9a; and a polyethylene terephthalate (PET) layer 9c that is provided on a surface of the aluminum layer 9b. In the illustrated example, the PP layer 9a is disposed on the inside of the first package 502, and the PET layer 9c is disposed on the outside of the first package 502. The first package 502 may be formed by preparing two sheets, with each sheet being made by bonding together the aluminum layer (aluminum foil) 9b, the PP layer (PP film) 9a, and the PET layer (PET film) 9c while the aluminum layer 9b is interposed between the PP layer 9a and the PET layer 9c, and thermally welding together the two sheets which are superimposed on top of each other with the PP layers 9a in contact with each other. The aluminum layer 9b may be formed using a vacuum deposition method. The PP layer 9a and the PET layer 9c may be formed using a film molding method such as an extrusion method.

Aluminum has water permeability and alcohol permeability lower than that of polypropylene that is the material of the adsorption container 100 and the cleaning containers 210 and 220. Since the first package 502 is a bag with the aluminum layer 9b, the first package 502 may have water permeability that is lower than that of the adsorption container 100 and the cleaning containers 210 and 220. In addition, the first package 502 may have alcohol permeability that is lower than that of the adsorption container 100 and the cleaning containers 210 and 220. For example, the water permeability and the alcohol permeability of the first package 502 may be 0 g/m²·day (at 40° C. and 90% RH).

Insofar as the material of the first package 502 has water permeability and alcohol permeability which are lower than that of the adsorption container 100 and the cleaning containers 210 and 220, the material of the first package 502 is not limited to a specific material. For example, instead of the aluminum layer 9b, a silica-deposited film may be used, or a layer made of ethylene-vinyl alcohol copolymer resin may be used.

The liquid holding material 504 contains alcohol. The liquid holding material 504 contains the same type of alcohol as that contained in the second cleaning solution 14 which is accommodated and sealed in the second cleaning container 220. The liquid holding material 504 contains methanol, ethanol, propanol, acetonitrile, isopropyl alcohol, or the like, and more preferably, ethanol. When alcohol is contained in the adsorption solution 10 that is accommodated and sealed in the adsorption container 100, the liquid holding material 504 may contain the same type of alcohol as that contained in the adsorption solution 10. When alcohol is contained in the first cleaning solution 12 that is accommodated and sealed in the first cleaning container 210, the liquid holding material 504 may contain the same type of alcohol as that contained in the first cleaning solution 12. The liquid holding material 504 may further contain water. The liquid holding material 504 is capable of containing and holding alcohol and water. The liquid holding material 504 may be adsorbent cotton through which alcohol and water permeate (which contains alcohol and water), or may be a porous material (specifically, sponge) through which alcohol and water permeate.

The liquid holding material 504 is capable of bringing an interior 506 of the first package 502 into a saturated alcohol-vapor state. In addition, the liquid holding material 504 is capable of bringing the interior 506 into a saturated water-vapor state. The term “saturated state” referred to here implies that alcohol or water is in a saturated vapor pressure state. The interior 506 may be brought into a saturated alcohol-vapor and water-vapor state by injecting liquid alcohol and liquid water into the interior 506 instead of providing the liquid holding material 504, which is not illustrated.

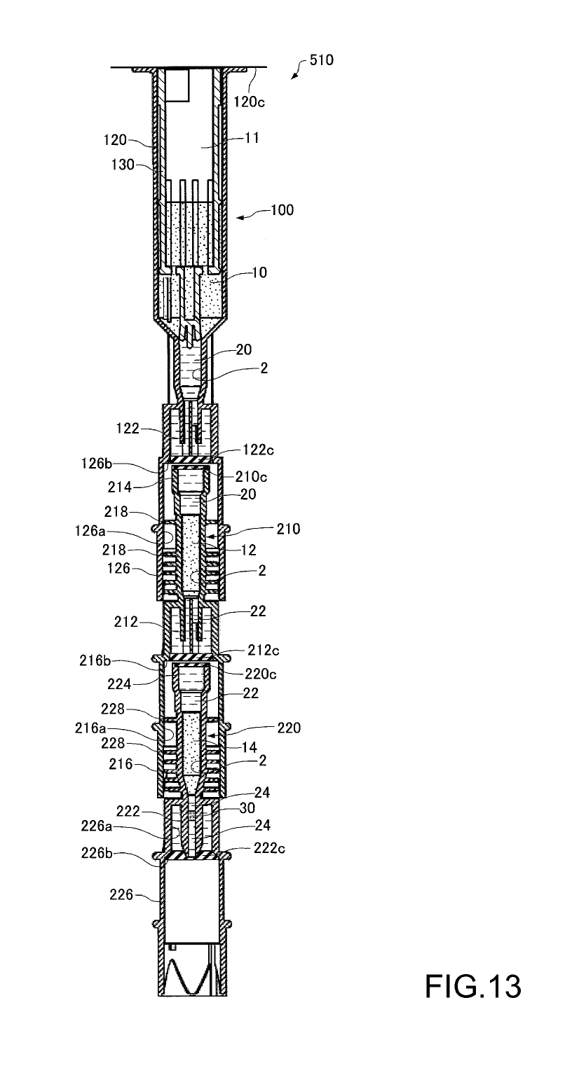

A first temporary assembly 510 in the interior 506 of the first package 502 is formed of the syringe portion 120 and the plunger portion 130 of the adsorption container 100, and the cleaning containers 210 and 220. FIG. 13 is a sectional view schematically illustrating the first temporary assembly 510, and illustrates the same section as that illustrated in FIG. 6.

As illustrated in FIG. 13, in the first temporary assembly 510, the flow passage 2 of the adsorption container 100 does not communicate with the flow passage 2 of the first cleaning container 210. In the first temporary assembly 510, the flow passage 2 of the first cleaning container 210 does not communicate with the flow passage 2 of the second cleaning container 220.

In the first temporary assembly 510, the adsorption insertion portion 122 of the adsorption container 100 is not inserted into the first receiving portion 214 of the first cleaning container 210. In the first temporary assembly 510, an internal wall 126a of the adsorption cover portion 126 is in contact with a flange 218 of the first cleaning container 210. Due to friction between the adsorption cover portion 126 and the flange 218, the syringe portion 120 of the adsorption container 100 is temporarily fixed to the first cleaning container 210 in a state where the syringe portion 120 is unlikely to move relative to the first cleaning container 210 in the vertical direction (the longitudinal direction of the flow passage 2).

The adsorption cover portion 126 is provided on the circumference of the adsorption insertion portion 122 of the adsorption container 100, and has an open bottom. The flange 218 protrudes outwards from an external wall of the first cleaning container 210, and has an annular shape in a plan view.

A film 120c is stuck to an upper end of the syringe portion 120 of the adsorption container 100. An upper end of the adsorption cover portion 126 of the adsorption container 100 is connected to an external wall of the adsorption insertion portion 122, and a lower end of the adsorption cover portion 126 extends while exceeding the adsorption insertion portion 122. The internal wall 126a of the adsorption cover portion 126 has an annular stepped portion 126b, the diameter of which is increased towards a lower side. The stepped portion 126b is positioned slightly below a lower end of the adsorption insertion portion 122, and a film 122c is stuck to a surface of the stepped portion 126b.

By means of the films 120c and 122c, the adsorption container 100 accommodates and seals the adsorption solution 10 which causes the nucleic acid to adsorb to the nucleic acid-binding solid-phase carrier (magnetic bead) 30, and the fluid (the first oil) 20 which is not mixed with the adsorption solution 10. In the illustrated example, air 11, the adsorption solution 10, and the first oil 20 are disposed sequentially from the film 120c to the film 122c. When the target nucleic acid is RNA, the adsorption solution 10 may contain alcohol (for example, ethanol), guanidine thiocyanate, and water. The ethanol contained in the adsorption solution 10 may have a concentration of greater than or equal to 40% by mass and less than or equal to 50% by mass. When the target nucleic acid is DNA, the adsorption solution 10 may not contain ethanol and guanidine thiocyanate, but contain guanidine hydrochloride and water.

In the first temporary assembly 510, the first insertion portion 212 of the first cleaning container 210 is not inserted into the second receiving portion 224 of the second cleaning container 220. In the first temporary assembly 510, an internal wall 216a of the first cover portion 216 is in contact with a flange 228 of the second cleaning container 220. Due to friction between the first cover portion 216 and the flange 228, the first cleaning container 210 is temporarily fixed to the second cleaning container 220 in a state where the first cleaning container 210 is unlikely to move relative to the second cleaning container 220 in the vertical direction.

The first cover portion 216 is provided on the circumference of the first insertion portion 212 of the first cleaning container 210, and has an open bottom. The flange 228 protrudes outwards from an external wall of the second cleaning container 220, and has an annular shape in a plan view.

A film 210c is stuck to an upper end of the first cleaning container 210. An upper end of the first cover portion 216 of the first cleaning container 210 is connected to an external wall of the first insertion portion 212, and a lower end of the first cover portion 216 extends while exceeding the first insertion portion 212. The internal wall 216a of the first cover portion 216 has an annular stepped portion 216b, the diameter of which is increased towards the lower side. The stepped portion 216b is positioned slightly below a lower end of the first insertion portion 212, and a film 212c is stuck to a surface of the stepped portion 216b.

By means of the films 210c and 212c, the first cleaning container 210 accommodates and seals the first cleaning solution 12 which cleans the magnetic bead 30 to which the nucleic acid adheres, and the fluids (the oils 20 and 22) which are not mixed with the first cleaning solution 12. In the illustrated example, the first oil 20, the first cleaning solution 12, and the second oil 22 are disposed sequentially from the film 210c to the film 212c. When the target nucleic acid is RNA, the first cleaning solution 12 may contain alcohol (for example, ethanol), guanidine hydrochloride, and water. The ethanol contained in the first cleaning solution 12 may have a concentration of greater than or equal to 50% by mass and less than or equal to 60% by mass. When the target nucleic acid is DNA, the first cleaning solution 12 may not contain ethanol, but contain guanidine hydrochloride and water.

In the first temporary assembly 510, a film 220c is stuck to an upper end of the second cleaning container 220. An upper end of the second cover portion 226 of the second cleaning container 220 is connected to an external wall of the second insertion portion 222, and a lower end of the second cover portion 226 extends while exceeding the second insertion portion 222. An internal wall 226a of the second cover portion 226 has an annular stepped portion 226b, the diameter of which is increased towards the lower side. The stepped portion 226b is positioned slightly below a lower end of the second insertion portion 222, and a film 222c is stuck to a surface of the stepped portion 226b.

By means of the films 220c and 222c, the second cleaning container 220 accommodates and seals the second cleaning solution 14 which cleans the magnetic bead 30 to which the nucleic acid adheres; the fluids (the oils 22 and 24) which are not mixed with the second cleaning solution 14; and the magnetic bead 30. In the illustrated example, the second oil 22, the second cleaning solution 14, the third oil 24, the magnetic bead 30, and the third oil 24 are disposed sequentially from the film 220c to the film 222c. When the target nucleic acid is RNA, the second cleaning solution 14 may contain alcohol (for example, ethanol), sodium chloride, and water. The ethanol contained in the second cleaning solution 14 may have a concentration of greater than or equal to 60% by mass and less than or equal to 70% by mass. When the target nucleic acid is DNA, the second cleaning solution 14 may not contain sodium chloride, but contain ethanol and water.

### 5-2. Second Accommodation Body

As illustrated in FIG. 11, the second accommodation body 600 includes a second package 602; a liquid holding material 604; the third cleaning container 230; and the elution container 300.

The second package 602 accommodates and seals (tightly seals) the liquid holing material 604, the third cleaning container 230, and the elution container 300. The shape of the second package 602 is not limited to a specific shape, and similar to the first package 502, the second package 602 may have a bag shape or a box shape. Insofar as the second package 602 is capable of accommodating and sealing the liquid holing material 604, the third cleaning container 230, and the elution container 300, the size of the second package 602 is not limited to a specific size. The second package 602 is made using the same method as used for the first package 502.

The second package 602 has water permeability that is lower than that of the third cleaning container 230 and the elution container 300. The second package 602 has alcohol permeability that is lower than that of the third cleaning container 230 and the elution container 300. Similar to the first package 502, the second package 602 is a bag with an aluminum layer. For example, the water permeability and the alcohol permeability of the second package 602 may be 0 g/m²·day (at 40° C. and 90% RH).

The liquid holding material 604 contains water. The liquid holding material 604 is capable of containing and holding water. The liquid holding material 604 may be adsorbent cotton through which water permeates (which contains water), or may be a porous material (specifically, sponge) through which water permeates. The liquid holding material 604 is capable of bringing an interior 606 of the second package 602 into a saturated water-vapor state. The interior 606 may be brought into a saturated water-vapor state by injecting liquid water into the interior 606 instead of providing the liquid holding material 604, which is not illustrated.

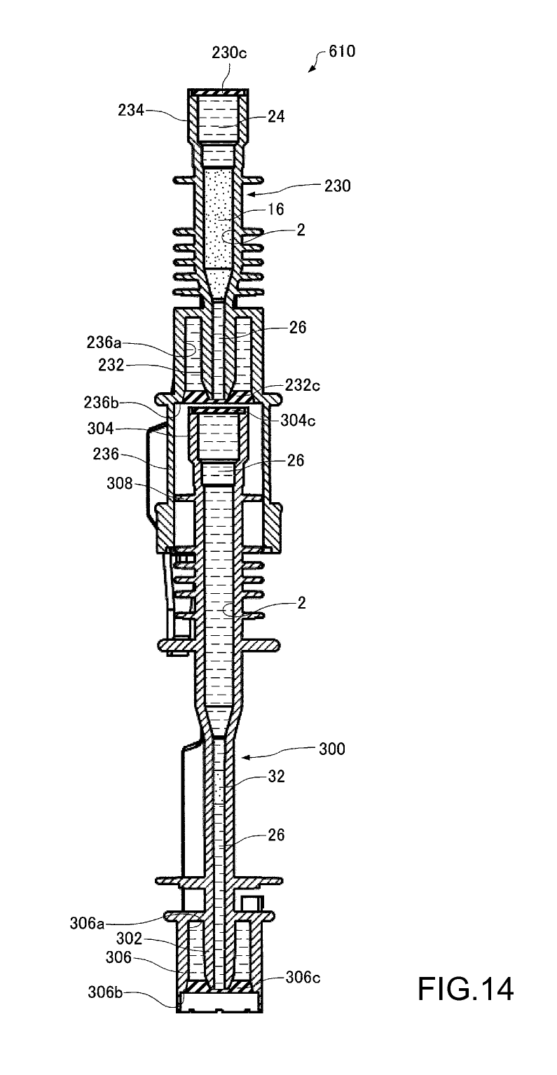

A second temporary assembly 610 in the interior 606 of the second package 602 is formed of the third cleaning container 230 and the elution container 300. FIG. 14 is a sectional view schematically illustrating the second temporary assembly 610, and illustrates the same section as that illustrated in FIG. 6.

As illustrated in FIG. 14, in the second temporary assembly 610, the flow passage 2 of the third cleaning container 230 does not communicate with the flow passage 2 of the elution container 300. In the second temporary assembly 610, the third insertion portion 232 of the third cleaning container 230 is not inserted into the elution receiving portion 304 of the elution container 300. In the second temporary assembly 610, an internal wall 236a of the third cover portion 236 is in contact with a flange 308 of the elution container 300. Due to friction between the third cover portion 236 and the flange 308, the third cleaning container 230 is temporarily fixed to the elution container 300 in a state where the third cleaning container 230 is unlikely to move relative to the elution container 300 in the vertical direction.

The third cover portion 236 is provided on the circumference of the third insertion portion 232 of the third cleaning container 230, and has an open bottom. The flange 308 protrudes outwards from an external wall of the elution container 300, and has an annular shape in a plan view.

A film 230c is stuck to an upper end of the third cleaning container 230. An upper end of the third cover portion 236 of the third cleaning container 230 is connected to an external wall of the third insertion portion 232, and a lower end of the third cover portion 236 extends while exceeding the third insertion portion 232. The internal wall 236a of the third cover portion 236 has an annular stepped portion 236b, the diameter of which is increased towards the lower side. The stepped portion 236b is positioned slightly below a lower end of the third insertion portion 232, and a film 232c is stuck to a surface of the stepped portion 236b.

By means of the films 230c and 232c, the third cleaning container 230 accommodates and seals the third cleaning solution 16 which cleans the magnetic bead 30 to which the nucleic acid adheres, and the fluids (the oils 24 and 26) which are not mixed with the third cleaning solution 16. In the illustrated example, the third oil 24, the third cleaning solution 16, and the fourth oil 26 are disposed sequentially from the film 230c to the film 232c. The third cleaning solution 16 may contain citric acid and water. The third cleaning solution 16 does not contain alcohol.

A film 304c is stuck to an upper end of the elution container 300. An upper end of the elution cover portion 306 of the elution container 300 is connected to an external wall of the elution insertion portion 302, and a lower end of the elution cover portion 306 extends while exceeding the elution insertion portion 302. An internal wall 306a of the elution cover portion 306 has an annular stepped portion 306b, the diameter of which is increased towards the lower side. The stepped portion 306b is positioned slightly below a lower end of the elution insertion portion 302, and a film 306c is stuck to a surface of the stepped portion 306b.

By means of the films 304c and 306c, the elution container 300 accommodates and seals the elution solution 32 which elutes the nucleic acid from the magnetic bead 30, and the fluid (the fourth oil 26) which is not mixed with the elution solution 32. In the illustrated example, the fourth oil 26, the elution solution 32, and the fourth oil 26 are disposed sequentially from the film 304c to the film 306c. The elution solution 32 may contain water.

### 5-3. Third Accommodation Body

As illustrated in FIG. 11, the third accommodation body 700 includes a third package 702; a drying agent 704; and the reaction container 400.

The third package 702 accommodates and seals (tightly seals) the drying agent 704 and the reaction container 400. The shape of the third package 702 is not limited to a specific shape, and similar to the first package 502, the third package 702 may have a bag shape or a box shape. Insofar as the third package 702 is capable of accommodating and sealing the drying agent 704 and the reaction container 400, the size of the third package 702 is not limited to a specific size. The third package 702 is made using the same method as used for the first package 502. In the illustrated example, the packages 502, 602, and 702 are separated from each other. The interior 506 of the package 502, the interior 606 of the package 602, and an interior 706 of the package 702 may have different volumes or the same volume.

The third package 702 has water permeability that is lower than that of the reaction container 400. The third package 702 has alcohol permeability that is lower than that of the reaction container 400. Similar to the first package 502, the third package 702 is a bag with an aluminum layer. For example, the water permeability and the alcohol permeability of the third package 702 may be 0 g/m²·day (at 40° C. and 90% RH).

The drying agent 704 is molecular sieve, silica gel, or the like, and with the absorption of water at a low humidity taken into consideration, the drying agent 704 preferably is molecular sieve. The molecular sieve is crystalline zeolite, and is a crystalline aluminosilicate material. The drying agent 704 is capable of absorbing water of the interior 706 of the third package 702.

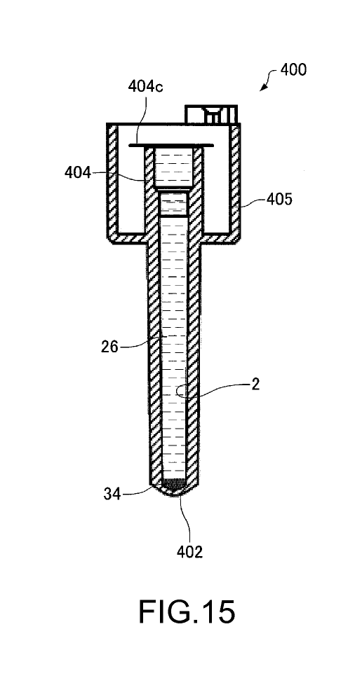

FIG. 15 is a sectional view schematically illustrating the reaction container 400 in the interior 706 of the third package 702, and illustrates the same section as that illustrated in FIG. 6.

As illustrated in FIG. 15, the reaction container 400 includes a reaction cover portion 405 that is provided on the circumference of the reaction receiving portion 404 and has an open top. A lower end of the reaction cover portion 405 is connected to an external wall of the reaction receiving portion 404, and an upper end of the reaction cover portion 405 extends while exceeding the reaction receiving portion 404. A film 404c is stuck to an upper surface of the reaction receiving portion 404.

By means of the film 404c, the reaction container 400 accommodates and seals the reagent 34 for performing a nucleic acid amplification reaction (for performing the PCR), and the fluid (the fourth oil 26) which is not mixed with the reagent 34. In the illustrated example, the reagent 34 is provided in the bottom portion 402 of the reaction container 400. The reagent 34 may be freeze dried. Specifically, the reagent 34 is dried by sublimating the moisture content in a decompressed state while being rapidly frozen at approximately −80° C. The fourth oil 26 may be an oil that is dehydrated by molecular sieve. The reagent 34 may be disposed in the fourth oil 26.

### 5-4. Assembly Method

An example of a method of assembling the cartridge set 7 will be described. First, the first temporary assembly 510 (refer to FIGS. 11 and 13) is taken out of the first package 502. The adsorption insertion portion 122 of the adsorption container 100 is inserted into the first receiving portion 214 of the first cleaning container 210 such that the adsorption container 100 is joined with the first cleaning container 210. The films 122c and 210c are torn apart by the adsorption insertion portion 122 and the first receiving portion 214. Accordingly, the flow passage 2 of the adsorption container 100 communicates with the flow passage 2 of the first cleaning container 210. The film 120c is torn apart by inserting a cotton swab into the adsorption solution 10 via the opening of the adsorption container 100 on which the cap 110 is mounted, with a specimen adhering to the cotton swab.

Subsequently, the first insertion portion 212 of the first cleaning container 210 is inserted into the second receiving portion 224 of the second cleaning container 220 such that the first cleaning container 210 is joined with the second cleaning container 220. The films 212c and 220c are torn apart by the first insertion portion 212 and the second receiving portion 224. Accordingly, the flow passage 2 of the first cleaning container 210 communicates with the flow passage 2 of the second cleaning container 220.

Subsequently, the second temporary assembly 610 (refer to FIGS. 11 and 14) is taken out of the second package 602. The third insertion portion 232 of the third cleaning container 230 is inserted into the elution receiving portion 304 of the elution container 300 such that the third cleaning container 230 is joined with the elution container 300. The films 232c and 304c are torn apart by the third insertion portion 232 and the elution receiving portion 304. Accordingly, the flow passage 2 of the third cleaning container 230 communicates with the flow passage 2 of the elution container 300, and the flow passages 2 from the adsorption container 100 to the elution container 300 communicate with each other.

Subsequently, the second insertion portion 222 of the second cleaning container 220 is inserted into the third receiving portion 234 of the third cleaning container 230 such that the second cleaning container 220 and the third cleaning container 230 are joined together. The films 222c and 230c are torn apart by the second insertion portion 222 and the third receiving portion 234. Accordingly, the flow passage 2 of the second cleaning container 220 communicates with the flow passage 2 of the third cleaning container 230, and the flow passages 2 from the adsorption container 100 to the reaction container 400 communicate with each other.

Subsequently, the reaction container 400 (refer to FIGS. 11 and 15) is taken out of the third package 702. The elution insertion portion 302 of the elution container 300 is inserted into the reaction receiving portion 404 of the reaction container 400 such that the elution container 300 and the reaction container 400 are joined together. The films 306c and 404c are torn apart by the elution insertion portion 302 and the reaction receiving portion 404. Accordingly, the flow passage 2 of the elution container 300 communicates with the flow passage 2 of the reaction container 400.

It is possible to obtain the cartridge 1 (refer to FIGS. 5 and 6, and the like) by assembling the cartridge set 7 via the aforementioned steps. A sequence of joining together the containers 100, 210, 220, 230, 300, and 400 is not limited to the sequence described in the example. A sequence of taking the containers 100, 210, 220, 230, 300, and 400 out of the packages 502, 602, and 702 is not limited to the sequence described in the example.

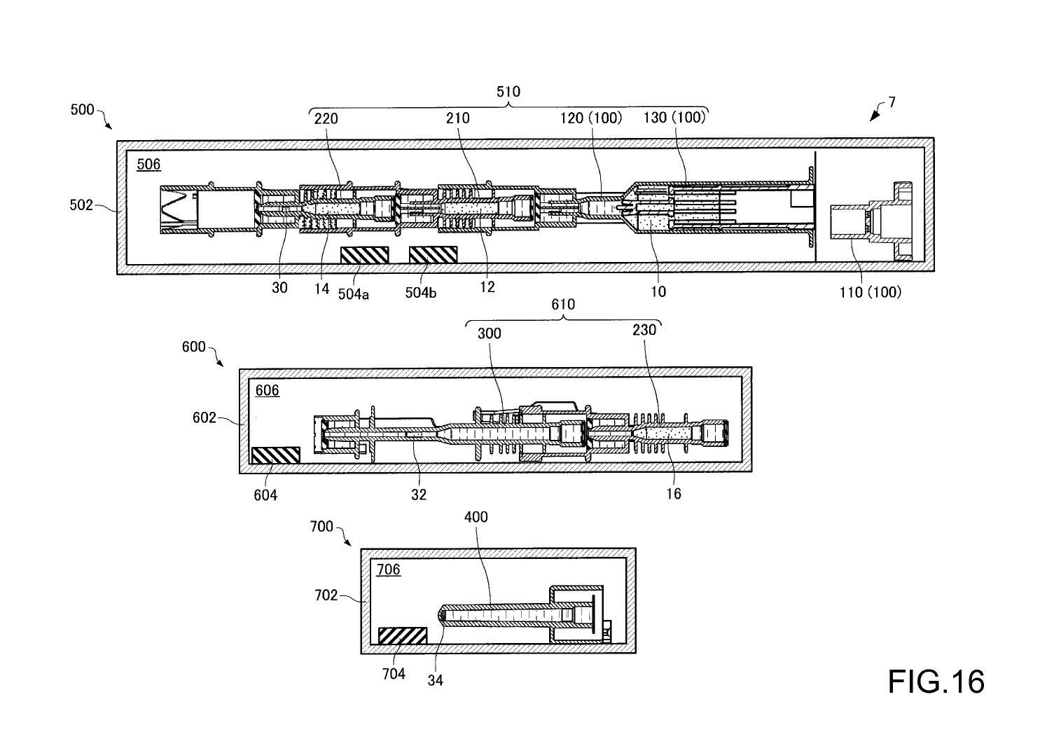

In the example described above, one liquid holding material 504 is accommodated and sealed in the first package 502; however, as illustrated in FIG. 16, two liquid holding materials 504a and 504b may be accommodated and sealed in the first package 502. The liquid holding material 504a may be adsorbent cotton containing alcohol. The liquid holding material 504b may be adsorbent cotton containing water.

In the example described above, the first temporary assembly 510 inside the first package 502 is formed of the adsorption container 100 and the cleaning containers 210 and 220, and the second temporary assembly 610 is formed of the third cleaning container 230 and the elution container 300; however, the containers 100, 210, 220, 230, and 300 do not form temporary assemblies, and may be separated from each other (not illustrated).

The third accommodation body (container accommodation body) 700 or the cartridge set 7 has the following characteristics.

The container accommodation body 700 includes the reaction container 400 that is accommodated and sealed in the third package 702 while accommodating and sealing the reagent 34, and a drying agent 704 that is accommodated and sealed in the third package 702. For this reason, in the container accommodation body 700, even if water enters the third package 702, since the drying agent 704 absorbs the water, the water is capable of being restricted from coming into contact with the reagent 34. Accordingly, even if the container accommodation body 700 is stored for a long time, water is capable of being restricted from coming into contact with the reagent 34.

In the cartridge set 7, the first temporary assembly 510, the second temporary assembly 610, and the reaction container 400 are respectively accommodated and sealed in separate packages. For this reason, even if the container accommodation body 700 is stored for a long time, water contained in the second cleaning solution 14 or the elution solution 32 is capable of being restricted from coming into contact with the reagent 34 via the flow passages 2.

For example, when water comes into contact with the freeze-dried reagent, enzymes contained in the reagent may deteriorate within a short time. In addition, when water comes into contact with the reagent for performing the nucleic acid amplification reaction, the reagent may be transformed into the form of molasses (the viscosity of the reagent is increased), and when the droplet containing the nucleic acid comes into contact with the reagent, the reagent may be unlikely to be dissolved, and the nucleic acid in the solution may be unlikely to be mixed with the reagent. As a result, the PCR (nucleic acid amplification reaction) may be restricted. For example, when approximately 0.1% by mass of water comes into contact with the reagent for performing diffusion-amplification reaction, the PCR is restricted.

In the container accommodation body 700, the third package 702 has water permeability that is lower than that of the reaction container 400. For this reason, in the container accommodation body 700, water contained in the second cleaning solution 14 or the elution solution 32, or water content or the like in the atmosphere is capable of being restricted from entering the reaction container 400 and coming into contact with the reagent 34 compared to when the reaction container is not accommodated and sealed in a package.

In the container accommodation body 700, the reagent is freeze dried. For this reason, in the container accommodation body 700, water content contained in the reagent 34 is capable of being reduced, and more preferably, the reagent 34 is capable of being prevented from containing water content.

In the container accommodation body 700, the reagent 34 is disposed in the fourth oil 26, and the fourth oil 26 is dehydrated. For this reason, in the container accommodation body 700, the fourth oil 26 is capable of being prevented from containing water.

In the container accommodation body 700, the third package 702 is a bag with the aluminum layer 9b. For this reason, in the container accommodation body 700, the water permeability of the third package 702 can be reduced.

The cartridge set 7 includes the second cleaning container 220 which is accommodated and sealed in the first package 502 while accommodating and sealing the second cleaning solution 14; the elution container 300 which is accommodated and sealed in the second package 602 while accommodating and sealing the elution solution 32; and the reaction container 400 which is accommodated and sealed in the third package 702 while accommodating and sealing the reagent 34. The first package 502 has water permeability that is lower than that of the second cleaning container 220, the second package 602 has water permeability that is lower than that of the elution container 300, and the third package 702 has water permeability that is lower than that of the reaction container 400. For this reason, in the cartridge set 7, the movement of water between the containers (for example, the movement of water between the containers 220 and 400, or the movement of water between the containers 300 and 400) can be restricted compared to when the containers are not accommodated and sealed in separate packages, respectively.

In the cartridge set 7, the interior 506 of the first package 502 is in a saturated alcohol-vapor and water-vapor state. For this reason, in the cartridge set 7, alcohol or water contained in the second cleaning solution 14 inside the second cleaning container 220 is capable of being restricted from permeating through the second cleaning container 220 and the first package 502, and evaporating. Similarly, the adsorption solution 10 inside the adsorption container 100, and the first cleaning solution 12 inside the first cleaning container 210 are capable of being restricted from evaporating.

For example, when alcohol or water contained in the second cleaning solution evaporates, air may enter the second cleaning container, and air bubbles may be produced in the flow passage of the second cleaning container. When the magnetic bead, to which the nucleic acid adheres, moves, the magnetic bead may be trapped on the boundary surface of an air bubble. When 0.8 μl (microliters) of alcohol or water evaporates, the flow passage may be blocked with produced air bubbles, and the amount of evaporation of alcohol or water to cause the blockage of the flow passage may be changed according to the diameter of the flow passage of the second cleaning container. As a result, the PCR may be adversely affected.

In the cartridge set 7, the interior 606 of the second package 602 is in a saturated water-vapor state. For this reason, in the cartridge set 7, water contained in the elution solution 32 inside the elution container 300 is capable of being restricted from permeating through the elution container 300 and the second package 602, and evaporating. Similarly, the third cleaning solution 16 inside the third cleaning container 230 is also capable of being restricted from evaporating.

When the amount of the elution solution is reduced due to the evaporation of the elution solution, the formation of a plug between the elution solution and the oil may become difficult. In particular, since a small amount of the elution solution is contained in the elution container, the evaporation of a portion of the elution solution causes the formation of the plug to become difficult. In addition, the concentration of the reagent in the PCR may be increased. As a result, the PCR may be adversely affected.

### 6. Modification Example of Cartridge Set

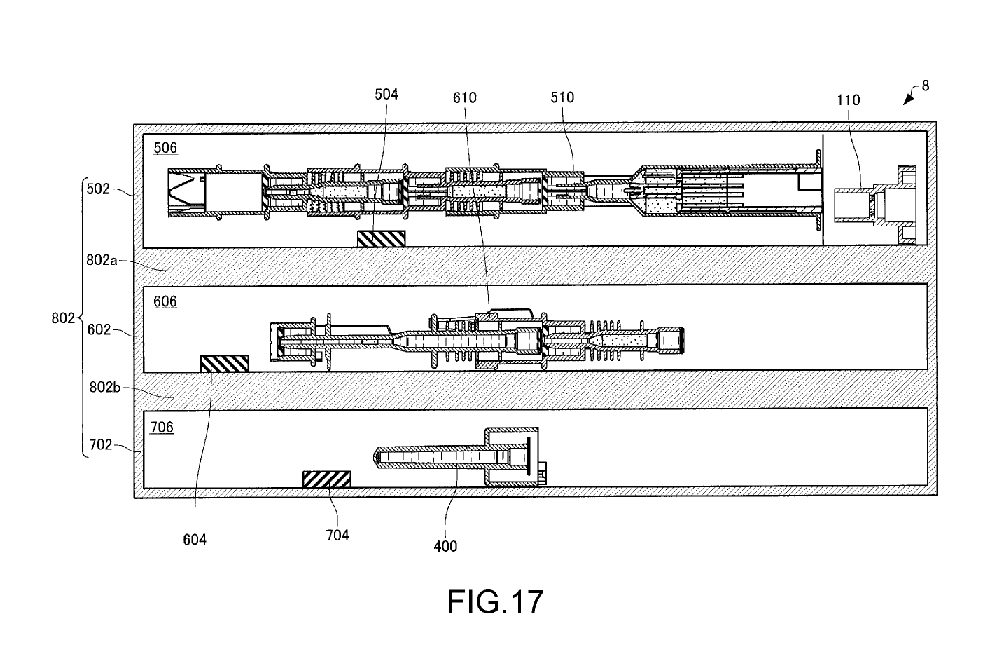

A cartridge set in a modification example of the embodiment will be described with reference to the drawing. FIG. 17 shows sectional views schematically illustrating a cartridge set 8 in the modification example of the embodiment. Hereinafter, the points of difference between the cartridge set 8 in the modification example of the embodiment and the cartridge set 7 in the example of the embodiment will be described, and the descriptions of the same points will be omitted.

As illustrated in FIG. 11, in the cartridge set 7, the packages 502, 602, and 702 are separated from each other. In contrast, as illustrated in FIG. 17, in the cartridge set 8, the packages 502, 602, and 702 are continuously formed.

Specifically, in the cartridge set 8, a large package 802 is thermally welded such that a first welded portion 802a and a second welded portion 802b are formed, and the packages 502, 602, and 702 are continuously formed. The temporary assemblies 510 and 610, and the reaction container 400 are respectively accommodated in the packages 502, 602, and 702 in such a way that the flow passages 2 thereof are disposed in the longitudinal direction. The interior 506 of the package 502, the interior 606 of the package 602, and the interior 706 of the package 702 have the same volume. In the illustrated example, the second package 602 is disposed between the first package 502 and the third package 702; however, the locations of disposition of the packages 502, 602, and 702 are not limited to specific locations.

In the cartridge set 8, the packages 502, 602, and 702 are continuously formed, and thus, the temporary assemblies 510 and 610, and the reaction container 400 are capable of being easily taken out of the packages 502, 602, and 702, respectively. In the cartridge set 7, when the temporary assemblies 510 and 610, and the reaction container 400 are taken out of the packages 502, 602, and 702, it is necessary to tear the packages 502, 602, and 702 one by one, that is, it is necessary to tear the packages 502, 602, and 702 a total of three times. In contrast, in the cartridge set 8, it is possible to easily take the temporary assemblies 510 and 610 and the reaction container 400 out of the packages 502, 602, and 702 by tearing the large package 802 only one time.

The invention is not limited to the embodiment, and various modifications can be made to the embodiment. The invention may also have substantially the same configuration (for example, a configuration in which functions, methods, and results are the same as in the embodiment, or a configuration in which objects and effects are the same as in the embodiment). The invention may also have a configuration in which a non-essential portion of the configuration in the embodiment is replaced. The invention may also have a configuration in which the same effects or the same object as in the embodiment can be obtained. The invention may also have a configuration in which well-known technology is added to the configuration in the embodiment.

The entire disclosure of Japanese Patent Application No. 2014-232504, filed Nov. 17, 2014 is expressly incorporated by reference herein.

What is claimed is:

1. A container accommodation body comprising:
a package;
a container that is accommodated and sealed in the package while accommodating and sealing a reagent for performing a nucleic acid amplification reaction; and
a drying agent that is accommodated and sealed in the package.

2. The container accommodation body according to claim 1,
wherein the package has water permeability that is lower than that of the container.

3. The container accommodation body according to claim 1,
wherein the reagent is freeze dried.

4. The container accommodation body according to claim 1,
wherein the drying agent is molecular sieve.

5. The container accommodation body according to claim 1,
wherein the container accommodates and seals a fluid that is not mixed with the reagent.

6. The container accommodation body according to claim 5,
wherein the reagent is disposed in the fluid, and
wherein the fluid is dehydrated.

7. The container accommodation body according to claim 1,
wherein the package is a bag with an aluminum layer.
